(19) United States
(12) Patent Application Publication
(10) Pub. No.: US 2013/0012796 A1
(43) Pub. Date: Jan. 10, 2013

(54) Smart device audio power harvesting glucose meter

(75) Inventors: Sanjeev Kak, Fremont, CA (US); Vipin Puri, Fremont, CA (US); Veeru Razdan, Sunnyvale, CA (US)

(73) Assignee: SV Square, Inc., Santa Clara, CA (US)

(21) Appl. No.: 13/135,512

(22) Filed: Jul. 7, 2011

(51) Int. Cl. A61B 5/145

(52) U.S. Cl. 600/365

(57) ABSTRACT

A smart device audio power harvesting glucose meter and a system and method for blood glucose measurement and monitoring. The glucose meter operates by harvesting the micro amp power from the audio port of a smart phone or other handheld smart device and does not need batteries or other power sources for its operation. A smart glucose monitoring application downloaded and embedded in the smart phone or other hand held smart device stores results of the glucose measurements in a personalized cloud database to be accessed by the user and shared with physicians, emergency personnel, friends, or family members if needed through automated phone calls, SMS/text, or emails. The smart glucose monitoring application is self learning and has an option for self ordering test strips based on the needs of the user.

### FIELD OF THE INVENTION

The present invention is generally related to glucose meters and blood glucose monitoring systems. More particularly, the invention relates to blood glucose meters integrated with handheld smart devices such as cell phones, smart phones, personal digital assistants (PDAs) and other similar devices.

### BACKGROUND OF THE INVENTION

The use of portable glucose meters for measuring and monitoring blood glucose levels has been in vogue for more than two decades. The majority of such glucose meters measure and display blood glucose levels on the small screen of the devices themselves when a specially treated test strip holding the blood droplet collected from a user is inserted through an aperture provided on the meter. Besides the measurements of blood glucose levels, current technology for these glucose meters is restricted to the display of features such as the date, time, and limited memory for retrieving present and past test results due to the limitations in memory and software available on the devices.

More recently, there have been several attempts to integrate glucose meters with smart phones such as the iPhone and related devices such as the iPod. These “Glugophones” as they are popularly known require the complete replacement of both the phone and the glucose meter that is integrated with the phone if either of them is damaged. The use of wireless technology to integrate glucose measuring devices with smart phones has also been explored by various groups. US Pat. Appl. Pub. No. 2010/0331645 describes a blood glucose meter comprising a measurement module and a wireless module which is operable to wirelessly communicate and transmit the result of blood glucose measurements from the glucose meter to a portable communication device such as a cellular phone, smart phone, or PDA. The glucose meter device of US Pat. Appl. Pub. No. 2010/0331654 is described as being wirelessly capable of communicating directly to a mobile phone network so as to provide data or messages stored on the meter. WO 2010/138817, describes a glucose monitor with only a test strip reader and wireless pairing with a smart phone for data communication.

The need for continuous glucose monitoring for diabetes management has necessitated the retrieval and storage of blood glucose measurements over a period of time lasting several hours, days, weeks and months. Instruments for continuous glucose monitoring generally collect the measurements and store them in a database for analysis by a health care provider. U.S. Pat. No. 7,912,674 to Killoren Clark et al., describes a method for processing, recording and storing a chronological sequence of measurements in a database and displaying at least three of the measurements simultaneously and graphically on a user interface such as a PDA, personal computer, a cellular phone, or smart phone.

The glucose meters in general and those described in the patent and publications operate by battery power or in the alternative from another power source such as an AC outlet. US Pat. Appl. Pub. No. 2010/0279418 describes a glucose device that is attached to the standard power connector interface of a handheld device such as an iPhone or iPod to communicate the glucose measurements to the handheld device. The device is either self powered by a rechargeable or replaceable internal battery or derives its power off the handheld device power connector interface.

The present invention is an improvement over the limited prior art relating to the use of blood glucose meters integrated with handheld smart devices such as cellular phones, the iPhone, PDA's and other similar devices. Unlike the described glucose meters in the prior art, the glucose meter of the present invention does not need batteries or other external power source for its operation. Neither does the glucose meter of the present invention use the power from the power connector interface of a smart phone or other handheld smart device for its operation. The glucose meter of the present invention operates solely by harvesting the power from the audio port of the handheld smart phone such as an iPhone or other handheld smart device.

### SUMMARY OF THE INVENTION

The present invention is a smart blood glucose meter that operates by harvesting the micro amp power from the audio port of a smart phone such as the Apple iPhone, Android phone, Windows phone 7 or other similar smart handheld devices. The glucose meter of the present invention does not use battery power or power from any other source except the already available power from the audio port of the smart phone or other hand held device for its operations. Thus the smart glucose meter of the present invention renders obsolete the need for batteries or other power sources for its operation. Since the power harvested by the glucose meter from the audio port of a smart phone or other smart hand held devices is in micro amps and therefore miniscule, it does not result in compromising the battery life of the handheld smart phone or device. The miniature size of the smart glucose meter of the invention allows it to be carried in a pocket or a keychain.

In an exemplary embodiment of the present invention, the smart phone or other smart hand held device has the appropriate smart glucose monitoring application embedded and available for use with the smart glucose meter of the present invention. In this embodiment, when the smart glucose meter of the invention is plugged into the audio port of the smart phone or other handheld device through the regular universal plug, the micro amp power from the audio port of the smart phone or other such device is harvested by the smart glucose meter to power itself In this embodiment, the energy harvesting circuit of the smart glucose meter is contained within the glucose meter chassis. The energy harvesting circuit is comprised of a 1:20 micro transformer to boost the input voltage, a FET bridge to rectify the AC signal to DC, parallel Shottky diodes to provide low-loss blocking to prevent the output filter capacitor from discharging through the FET bridge, and an optional LED with current-limiting resistor to provide a visual power indicator.

In the exemplary embodiment of the smart glucose meter of the present invention, when the meter is plugged into the audio port of the iPhone, Android phone, Windows 7 phone or other similar handheld smart devices, the device will detect the audio port connection of the meter and automatically start the smart glucose meter application software that is downloaded and embedded in the hand held device. If the application has not been downloaded to the device, once the smart glucose meter is plugged into the audio port of the device, the user will be prompted or given a choice to download the application before proceeding with the next steps. Once the application is downloaded, the user of the smart glucose meter follows the instructions displayed on the smart phone screen or device to take the next step. In this embodiment, once the smart glucose meter is powered by harvesting the power from the audio port of the smart phone and activated, the smart phone or other device displays the presence of the glucose meter on its screen and prompts the user to insert a test strip into the designated aperture on the meter. After the test strip is inserted, the smart phone displays a command for the user to deposit a blood sample on to the test strip of the glucose meter. Once the user deposits a blood sample on the test strip of the meter, the testing is commenced and the results are displayed on the smart phone or device screen.

In this exemplary embodiment of the smart glucose meter of the present invention, the system uses an algorithm to automatically determine and record the various parameters related to the blood glucose test such as, time of day when the test was done, the type of test whether fasting, after meal, before bedtime, or random testing. The cumulative results are stored in a personalized cloud database and can be accessed by an authorized user from a phone, a desktop application such as Windows, Mac, Linux etc., or the website. In this embodiment, the powerful user management feature of the application allows the user to configure secure settings so that results are not altered by a read only user. In this embodiment, the user is also able to set up a phone call, email, or SMS reminder to them to do the blood glucose testing.

In yet another embodiment of the present invention, the system uses an algorithm to set up notifications of the test results to health care or emergency providers, friends, or family via, phone call, email or SMS. The system can be configured by the user to call his/her physician, an emergency number, friend, or family member and provide a Map/Geo location so that help is directed to that location. In this embodiment, the system uses the GPS technology embedded in the smart phone or other smart device to provide the Map/Geo location to the emergency provider, physician, friend, or family member.

In yet another exemplary embodiment of the present invention, the system of the smart glucose meter, will have an option set up for self ordering test strips based on an algorithm that relates to the model number of the smart glucose meter, the number of strips used, the number of tests performed per day, number of tests performed since last order, total number of days left before inventory of the test strips exhausts etc.

An embodiment of the smart glucose monitoring system of the present invention allows the user to analyze stored data selected by user and suggests menu or calorie options based on test results.

In a related embodiment of the present invention, the smart device audio power harvesting mechanism, circuit and hardware of the invention can also be incorporated into additional meters and devices used for point of care testing of other blood markers such as Prothrombintine/INR (International normalized ratio), Rapid HIV Test, CRP (Inflammatory Marker) Test, Tests for Cholesterol, Electrolytes, and HB A1C (hemoglobin).

These and other features and advantages of the present invention through its embodiments will become obvious to one skilled in the arts when viewed in conjunction with the ensuing description of the drawings, detailed description of the invention and the claims that follow.

### BRIEF DESCRIPTION OF THE DRAWINGS

FIG. 1 shows a perspective view of the coupling of the smart glucose meter with the smart phone audio port.

FIG. 2 shows a schematic diagram of the power harvesting and communication circuit.

FIG. 3 is a flow chart representing the initial power harvesting steps when the smart glucose meter is plugged into the audio port of a smart phone.

FIG. 4 is a flow chart illustrating the choices presented to the user when the smart monitoring application is started after the smart glucose meter is plugged into the audio port of the smart phone.

FIG. 5 illustrates the steps involved in the measurement of blood glucose by the smart glucose meter and the presentation of the results on the smart phone screen.

FIG. 6 highlights the exemplary features of the automatic smart monitoring application of the glucose monitoring system of the invention.

FIG. 7 is a flow diagram of the options available to the user to analyze the data from the blood glucose measurements based on the various parameters.

FIG. 8 illustrates the type of settings available to the user of the smart glucose monitoring system.

FIG. 9 is the representation of algorithm 1 for the type of test done using the smart glucose monitoring system.

FIG. 10 is the representation of algorithm 2 for the intelligent strip ordering system.

FIG. 11 is the representation of algorithm 3 for the call/email/text results.

FIG. 12 is the representation of algorithm 4 for the phone call reminders on tests.

### DETAILED DESCRIPTION OF THE INVENTION

The present invention is a blood glucose monitoring system comprising a smart blood glucose meter that is powered by harvesting the micro amp power from the audio port of a smart phone or other handheld smart device. By harvesting the power from the audio port of the smart device, the smart glucose meter obviates the need for using batteries or power from other sources through cables or other connections for its operation and in addition does not compromise the battery life of the smart phone or other handheld smart device to which it is plugged into when testing and relaying the blood glucose measurements to the device.

The blood glucose monitoring system of the present invention has the appropriate hardware configured in the smart glucose meter of the invention and software applications configured on the smart phone or other handheld smart device to allow for the analysis and monitoring of the blood glucose levels of the user, by suggesting test types to be used, self-ordering test strips, menu and calorie options based on test results, notifying emergency, physician, friend, or family member through, phone call, SMS/texting, or email of the geo location of the user if needed.

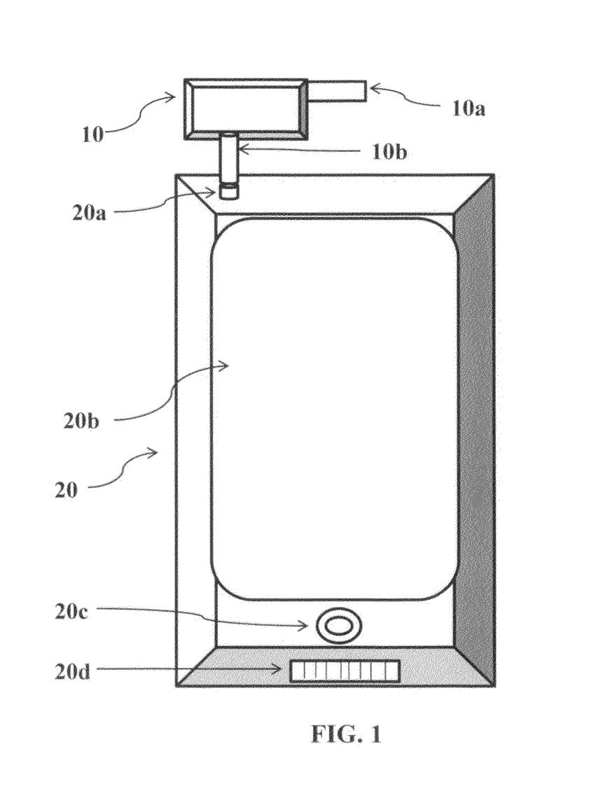

Referring now to the figures, more particularly to FIG. 1, there is shown a perspective view of the coupling of the smart glucose meter 10 of the present invention to a smart phone 20. The smart glucose meter 10 is plugged into the audio port 20a of the smart phone 20 through a regular universal plug 10b and starts harvesting the micro amp power from the audio port 20a of the smart phone 20. The smart phone 20 detects the smart glucose meter 10 when it is plugged into the phone audio port 20a through the meter's regular universal plug 10b and immediately starts the smart glucose monitoring application embedded within the smart phone 20. If the application is not downloaded and present on the smart phone 20, the system will alert the user that the application needs to be downloaded in order to proceed with the next steps. The application provides the user with several choices for the type of test to be performed and asks the user to insert the test strip 10a into the aperture provided on the smart glucose meter 10. Once the test strip 10a is inserted into the aperture on the smart glucose meter 10, the smart glucose monitoring application prompts the user to place a sample of blood on the test strip 10a. The placing and impregnation of the blood onto the test strip 10a starts the measurement of the blood glucose level by the smart glucose meter 10 and the result is displayed on the smart phone screen 20b. 20c in this figure references the start button for the smart phone 20 and 20d references the main power connector interface of the smart phone 20.

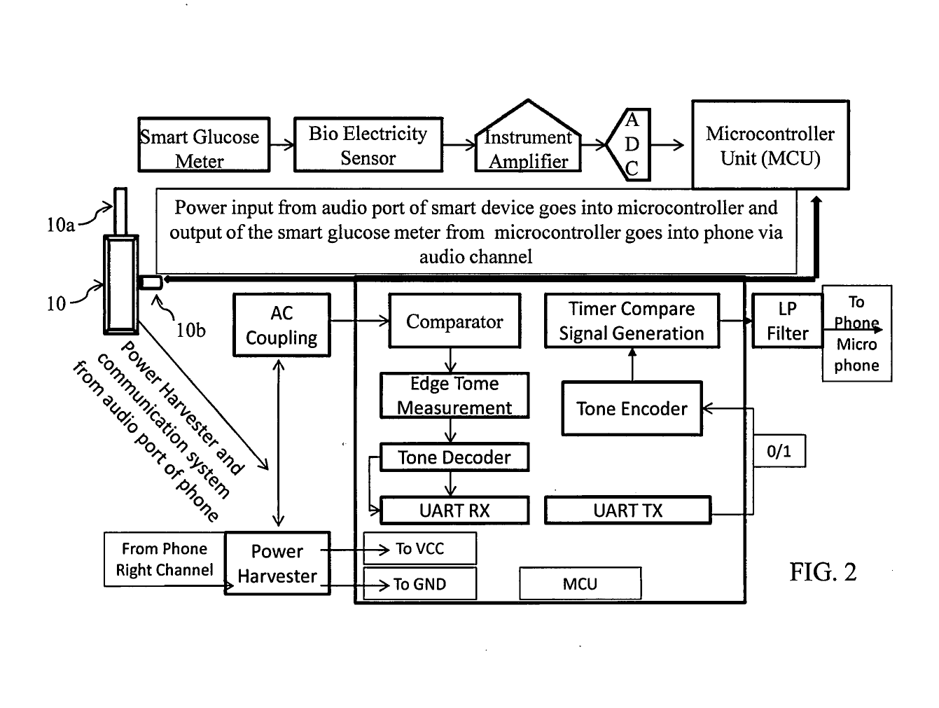

FIG. 2 is a schematic diagram of the integrated power harvesting and communication circuit. Once the smart glucose meter 10 is coupled with the smart phone or other smart handheld device, through the regular universal plug 10b, the power input from the audio port of the smart device goes into the microcontroller embedded in the smart glucose meter and the output of the smart glucose meter 10 from the microcontroller goes into the smart phone or other device via the audio channel of the device. Test strip 10a receives the blood sample and the test results are displayed on the smart phone or other smart device screen.

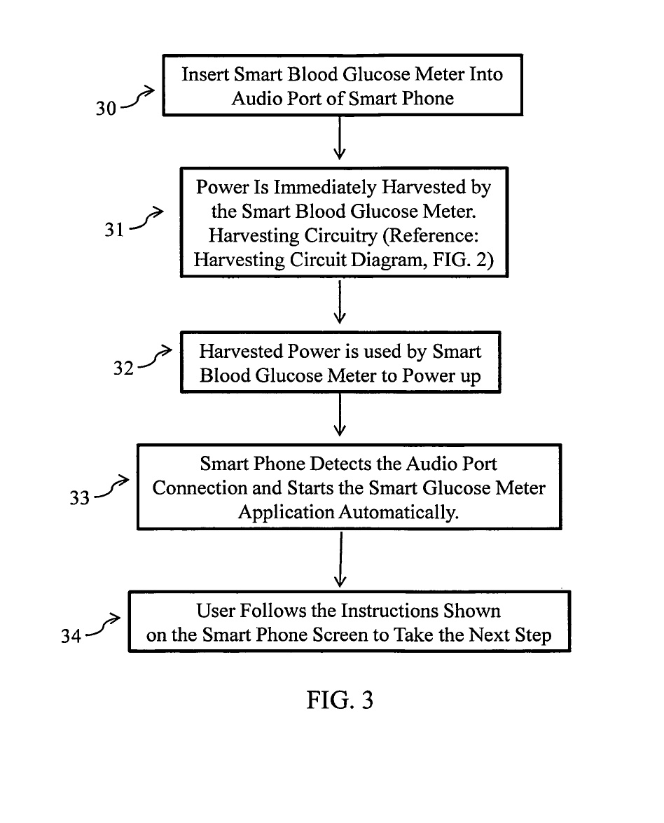

FIG. 3 is a flow chart representing the initial power harvesting steps when the smart glucose meter is plugged into the audio port of a smart phone. The first step 30 is the plugging in or insertion of the smart glucose meter into the audio port of the smart phone. Power is immediately harvested 31 by the smart glucose meter using the harvesting circuitry diagram shown in FIG. 2. Harvested power 32 is used by the smart glucose meter to power up. Once the smart glucose meter is powered up, the smart phone detects the audio port connection and starts the smart glucose meter application automatically 33. User follows the instructions shown on the smart phone screen to take the next steps 34.

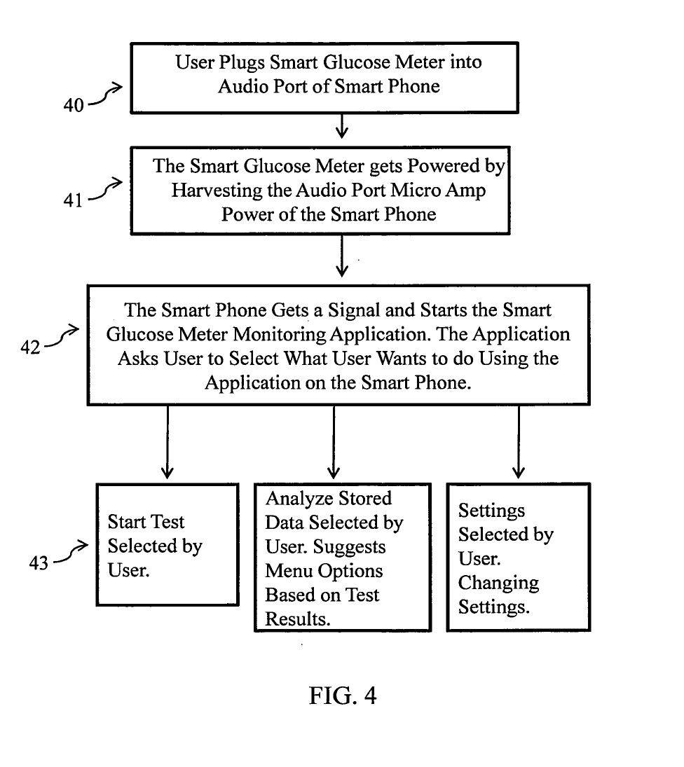

FIG. 4 is a flow chart illustrating the choices presented to the user when the smart glucose meter monitoring application is started after the smart glucose meter is plugged into the audio port of the smart phone. First, 40 the user plugs the smart glucose meter into the audio port of the smart phone. Second, 41 the smart glucose meter gets powered by harvesting the audio port micro amp energy of the smart phone. Third, 42 the smart phone gets a signal and starts the smart glucose meter monitoring application. The application asks user to select what the user wants to do using the application presented on the smart phone. Fourth, 43 the user is given a choice between starting a selected test, analyzing stored data selected by the user including suggestions for menu or calorie choices based on test results, or changing settings selected by the user. If the user does not want to start a test, the user may analyze stored data or conduct other functions using the application without having the smart glucose meter plugged into the smart phone.

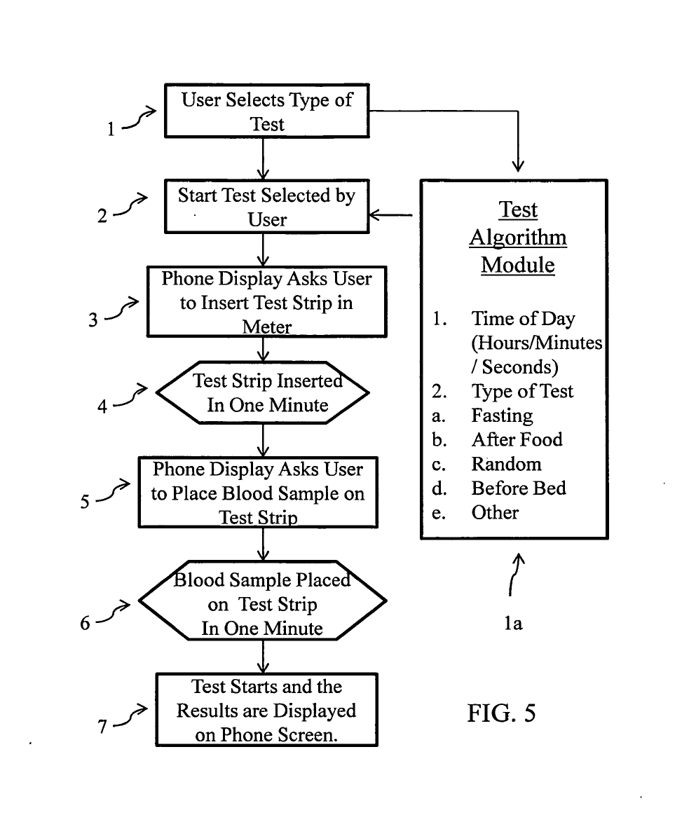

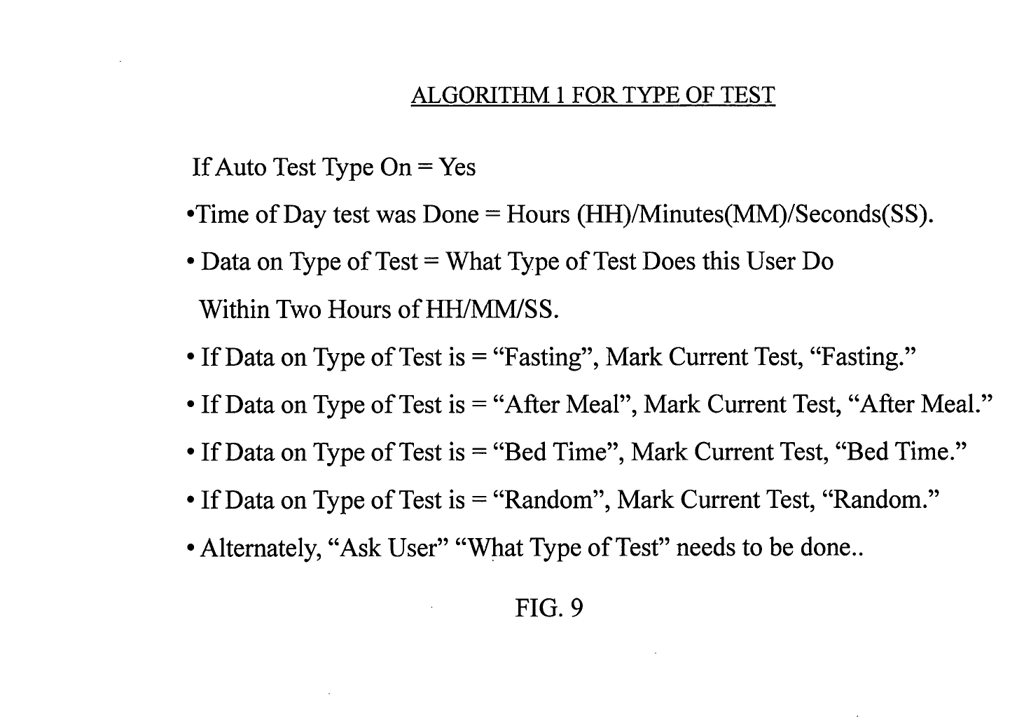

FIG. 5 illustrates the steps involved in the measurement of blood glucose by the smart glucose meter and the presentation of the results on the smart phone screen. Step 1, user selects type of test using algorithm 1a, FIG. 9. Step, 2, start test selected by user. Step 3, phone display asks user to insert test strip in the meter. Step 4, test strip is inserted within x seconds. Step 5, phone display asks user to place blood sample on the test strip. Step 6, blood sample is placed on the test strip in x seconds. Step 7, test of the blood sample starts and the glucose measurement results are displayed on the smart phone screen.

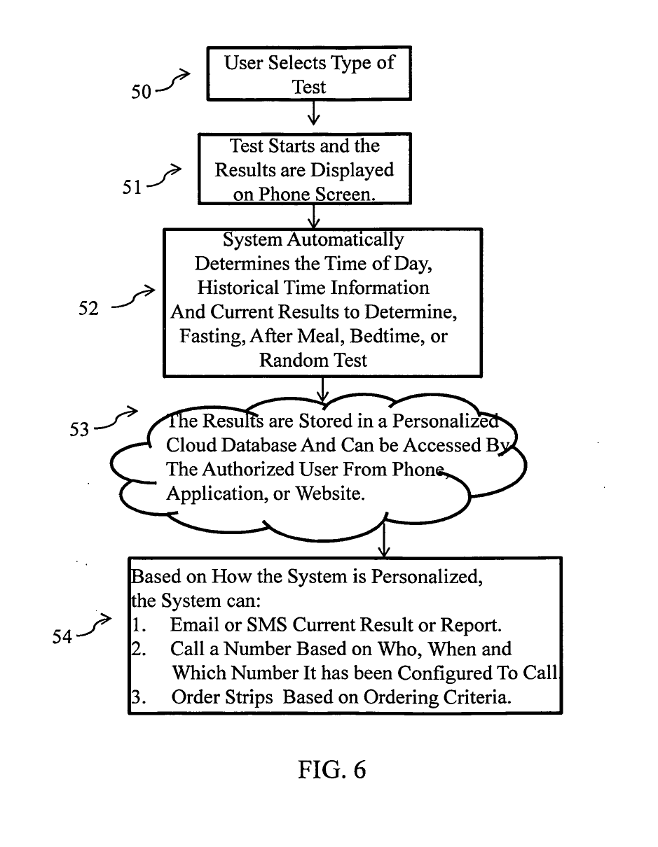

FIG. 6 highlights the exemplary features of the automatic smart monitoring application of the glucose monitoring system of the invention. User selects type of test 50. Test starts and the results are displayed on the phone screen 51. System automatically determines the time of day, historical time information and current results to determine, fasting, after meal, bedtime, or random test 52. The results are stored in a personalized cloud database 53 and can be accessed by the authorized user from a phone, a desktop application such as, Windows, Mac, Linux etc., or a website. Based on how the system is personalized, 54 the system can email or SMS/text current result or report, call a number based on who when and which number it has been configured to call, or order strips based on ordering criteria.

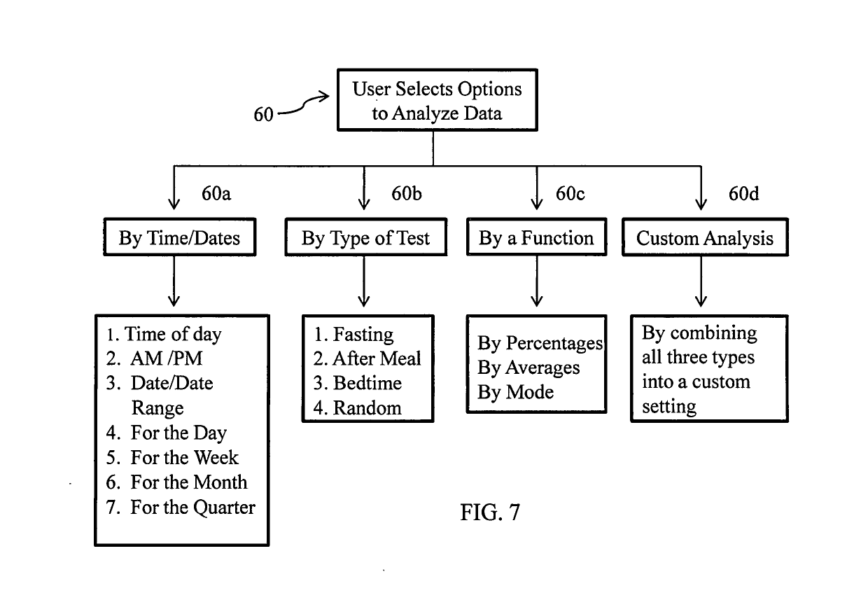

FIG. 7 is a flow diagram of the options available to the user to analyze the data from the blood glucose measurements based on the various parameters. User selects options to analyze data 60 by time or dates 60a, by type of test 60b, by a function 60c, or custom analysis 60d. The time of day and date analysis can be for AM or PM, dates and date ranges. The type of test analysis can be for fasting, after meal, bedtime or random testing. Analysis by function can pertain to percentages, averages or modes of the measurement results. The user also has the ability to conduct a custom analysis by combining all three types of analysis into a custom setting.

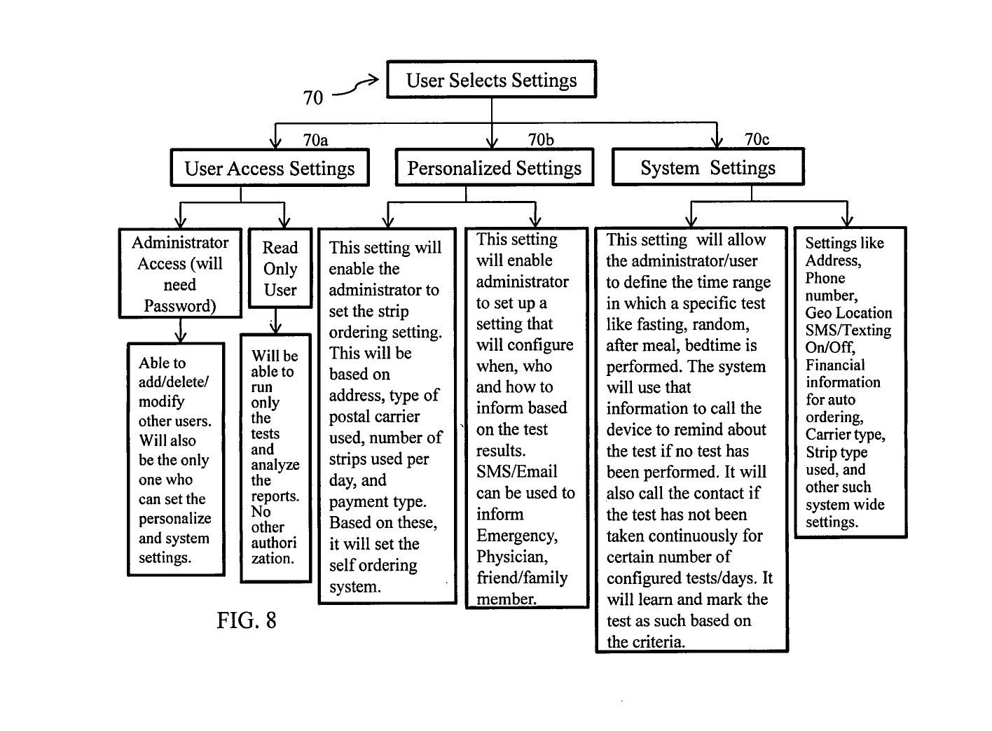

FIG. 8 illustrates the type of settings available to the user of the smart glucose monitoring system. User selects settings 70. The settings available to the user are user access settings 70a, personalized settings 70b and system settings 70c. The user access settings 70a has two settings, an administrator access setting and a read only user access setting. The administrator access setting requires authentication through a password and allows only the administrator user to set the personalized and system settings. With this setting, the administrator can add or delete other users from accessing the data stored in the database. The read only user setting allows only testing and analyzing the results. The personalized settings 70b will enable the administrator user to set the strip self ordering setting based on address, type of postal carrier used, number of strips used per day, and payment type. The personalized setting 70b also allows the administrator user to set up a setting that will configure when, who, which number to call and other ways to inform third parties based on the test results. Phone, SMS/text, or email can be used to contact and inform emergency, physician, friend, or family member. The system settings 70c, allows the administrator user to define the time range in which a specific test such as fasting, after meal, bedtime, or random is to be performed. The system will use that information to call the smart phone or other such device of the user to remind about the test if no test has been performed. The system will also call the designated contact if the test has not been taken continuously for certain number of configured tests per day or days. The system has the ability to self learn over time the time of day the readings are taken by the user or patient and based on the readings will try to intelligently assess whether the test is for fasting, after meals, bedtime, or random. The system will allow the user to change the settings for the various tests. The system settings 70c also allows the administrator, user to set up addresses, phone numbers, geo location, SMS/texting on/off, financial information for auto ordering, carrier type, strip type used, and other such system wide settings.

FIG. 9 is the representation of algorithm 1 for the type of test done using the smart glucose monitoring system. The algorithm details the time of day the test was or is done in hours, minutes, or seconds, test type such as, fasting, after meals, bedtime, random etc., the specific user does within a certain number of hours, data on the type of testing, fasting, after meal, bedtime, or random and alternately asks user what type of test needs to be done.

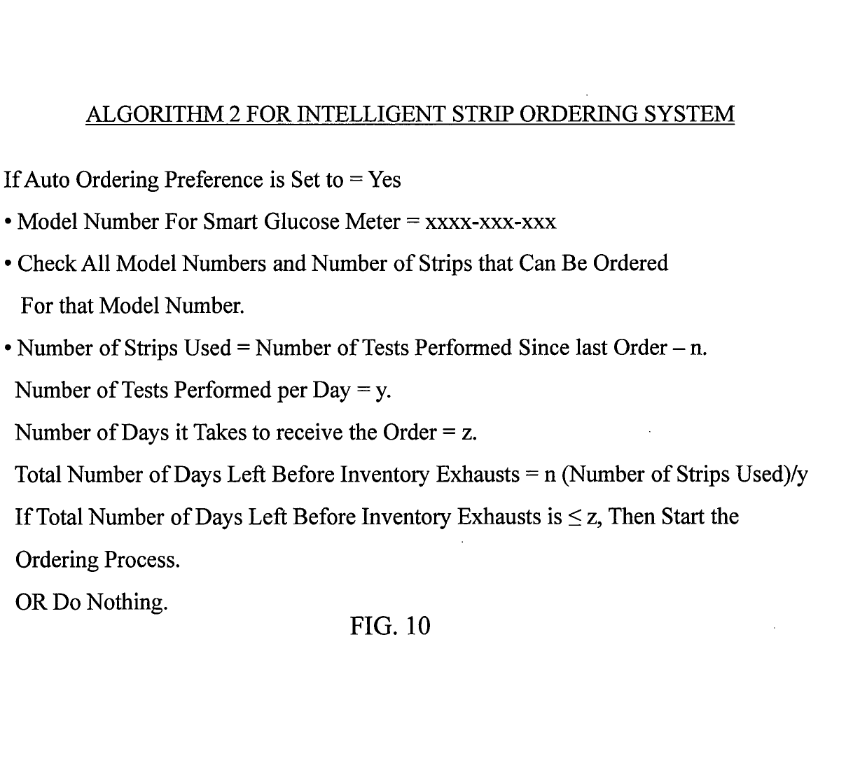

FIG. 10 is the representation of algorithm 2 for the intelligent strip ordering system. If auto strip ordering preference is set to yes, the system checks the model number for the smart glucose meter currently used and the number of strips that can be ordered for that model number. Before placing the order for the strips, the system checks on the number of tests performed since last order, number of tests performed per day, number of days it takes to receive an order, total number of days before inventory exhausts. If the total number of days left before inventory exhausts is less than or equal to the number of days it takes to receive the order, then the system starts the ordering process.

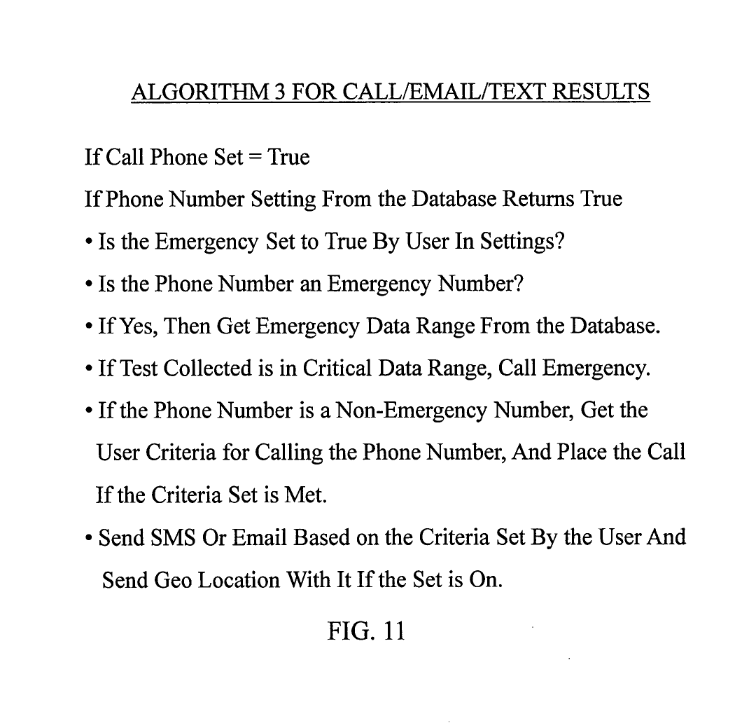

FIG. 11 is the representation of algorithm 3 for the call/email/text results. Using this algorithm, the system calls a phone number listed for emergency, physician, friend, or family member if the test results are in the critical data range necessitating a phone call, SMS/text, or email to inform the parties as well as provide the geo location where the call, email, or messages are from so that help can be sent immediately to that geo location where the user, patient is.

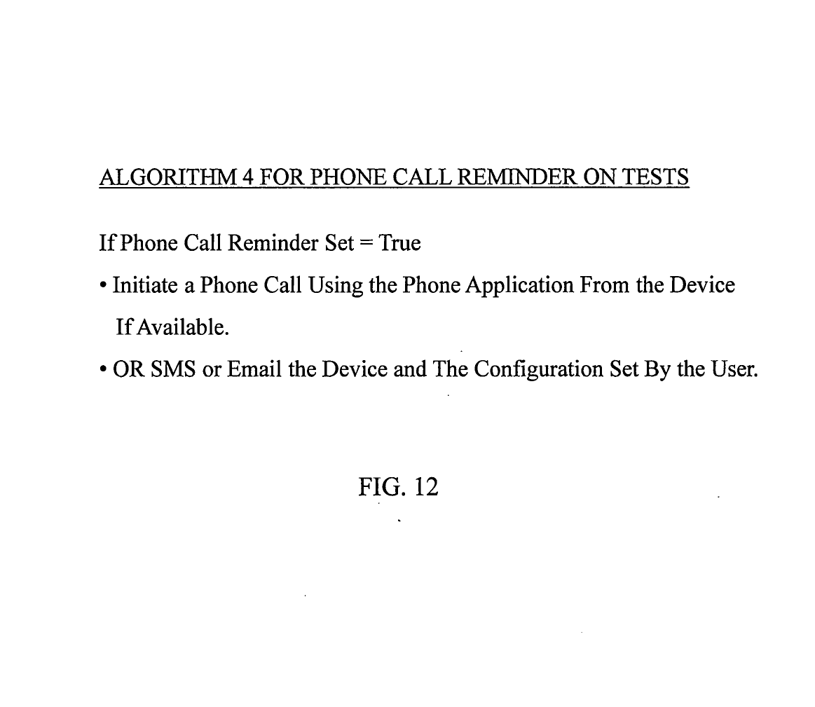

FIG. 12 is the representation of algorithm 4 for the phone call reminders on tests. The system through this algorithm sets up a reminder phone call, SMS/text or email to remind the user to perform the test if the user forgets to do it at the specified time of day, week, and month etc.

The foregoing description of the present invention through its figures and preferred embodiments should not be construed to limit the scope of the invention. It should be understood and obvious to one skilled in the art that the embodiments of the invention thus described may be further modified without departing from the spirit and scope of the invention as forth in the claims that follow.

What is claimed is:

1. A blood glucose monitoring system comprising:
a blood glucose measurement meter configured to plug into the audio port of a portable smart phone or other handheld smart device;
said blood glucose measurement meter housing an energy harvesting circuit to harvest the micro amp power from the said audio port of the said smart phone or other hand held smart device to power the meter;
said blood glucose measurement meter having an aperture to receive a test strip; and
a smart glucose monitoring application embedded within the smart phone or other handheld smart device.

2. The blood glucose monitoring system of claim 1 wherein the blood glucose measurement meter is portable in a pocket or on a key chain.

3. The blood glucose monitoring system of claim 1 wherein the blood glucose measurement meter does not need batteries or other sources of power for its operation.

4. The blood glucose monitoring system of claim 1 wherein the portable hand held smart device is a cell phone, an iPhone, an iPod, an Android phone, a Windows 7 phone, a PDA (personal digital assistant).

5. The blood glucose monitoring system of claim 1 wherein the energy harvesting circuit is housed within the blood glucose measurement meter and additionally comprises of a microcontroller, a bioelectric sensor and other needed circuitry to harvest the power from the audio port of the smart phone or other handheld smart device.

6. The blood glucose monitoring system of claim 1 wherein the smart blood glucose monitoring application embedded in the smart phone or other handheld smart device displays the blood glucose measurements generated by the blood glucose meter on the smart phone or handheld smart device screen.

7. The blood glucose monitoring system of claim 1 wherein the smart glucose monitoring application automatically determines, time of day in hours, minutes, seconds, as well as historical time, type of test whether fasting, after meal, bedtime, or random.

8. The blood glucose monitoring system of claim 1 wherein, the smart blood glucose monitoring application stores the daily, weekly, monthly and historical blood glucose measurement results in a personalized cloud database capable of being accessed by the authorized user from a phone, a desktop application such as Windows, Mac, Linux etc., or website.

9. The blood glucose monitoring system of claim 1 wherein the personalized stored data can be shared with a physician or emergency personnel, friend, or family member by phone, email, or SMS/text.

10. The blood glucose monitoring system of claim 1 wherein the system can be configured by the user to call a physician, an emergency number, friend, or family member and provide a map/geo location by GPS or other such technology embedded in the smart phone or other handheld smart device so that help is directed to that location.

11. The blood glucose monitoring system of claim 1 wherein the system can be configured to set up reminders to the user about testing through the smart phone or other handheld smart device of the user.

12. The blood glucose monitoring system of claim 1 wherein the system is configured to self order test strips based on need and inventory.

13. The blood glucose monitoring system of claim 1 wherein the smart glucose monitoring system analyzes the test results and suggests menu or calorie options depending on the test results.

14. The blood glucose monitoring system of claim 1 wherein the energy harvesting circuit to harvest the micro amp power from the audio port of a smart phone or other hand held smart device to power the meter can also be used to power other meters and devices used for point of care testing of blood markers such as Prothrombintine/INR (International normalized ratio), Rapid HIV Test, CRP (Inflammatory Marker) Test, Tests for Cholesterol, Electrolytes, and HB A1C (hemoglobin).

15. A method of measuring blood glucose levels, the method comprising:
inserting a smart glucose meter into the audio port of a smart phone or other handheld smart device through a regular universal plug configured on the smart glucose meter;
harvesting the micro amp energy from the audio port of the smart phone or other handheld smart device;
powering up the smart glucose meter using the micro amp energy from the audio port of the smart phone or other handheld smart device;
inserting a test strip into the aperture of the smart glucose meter;
placing a blood sample on the test strip;
measuring the blood glucose level by the smart glucose meter;
transmitting the blood glucose measurement to the smart glucose monitoring application embedded within the smart phone or hand held smart device; and
displaying the blood glucose measurement on the smart phone or other handheld smart device screen.

16. The method of claim 15 wherein the smart glucose monitoring application on the smart phone or other handheld smart device stores the results in a personalized cloud database for access by the authorized user from a phone, a desktop application such as Windows, Mac, Linux etc., or website.

17. The method of claim 15 wherein the results stored in the personalized cloud database can be shared with a physician, emergency personnel, friend, or family member through phone call, email, or SMS/text.

18. A smart glucose meter comprising:
a blood glucose meter;
a power harvesting circuit within the meter for harvesting the micro amp power from the audio port of a smart phone or other handheld smart device;
a regular universal plug to insert and connect the smart glucose meter with the audio port of a smart phone or other handheld smart device to harvest the micro amp power from the audio port of the device;
a microcontroller, bio electric sensor and other needed circuitry embedded within the smart glucose meter to harvest the power from the audio port of the smart phone or other handheld smart device;
a test strip aperture for inserting a test strip for measuring the blood glucose;
transmitting the test results to a smart phone or other handheld smart device; and
displaying the test results on the smart phone or other handheld smart device screen through a smart glucose monitoring application embedded within the device.
